(19) United States
(12) Patent Application Publication
(10) Pub. No.: US 2026/0311883 A1
(43) Pub. Date: Oct. 8, 2026

(54) USING NEURAL DATA FROM INFANCY TO FORECAST FUTURE DEVELOPMENTAL OUTCOMES IN PRETERM CHILDREN

(71) Applicant: The Chinese University of Hong Kong, Shatin, New Territories, Hong Kong SAR (CN)

(72) Inventors: Patrick Chun Man WONG, Tai Po, N.T., Hong Kong SAR (CN); Nikolay NOVITSKIY, Yung Shue Wan, Lamma Island (CN); Xiujuan GENG, Ma On Shan, N.T., Hong Kong SAR (CN); Hiu Ying CHAN, Shatin, N.T., Hong Kong SAR (CN); Hugh Simon Hung San LAM, Shatin, N.T., Hong Kong SAR (CN); Ting Fan LEUNG, Shatin, N.T., Hong Kong SAR (CN)

(21) Appl. No.: 19/476,145

(22) PCT Filed: Apr. 16, 2024
(86) PCT No.: PCT/CN2024/087906

§ 371 Date: Oct. 16, 2025

Related U.S. Application Data
(60) Provisional application No. 63/461,204, filed on Apr. 21, 2023.

(51) Int. Cl. G16H 50/30; G16H 50/20; G16H 50/70

(52) U.S. Cl. CPC G16H 50/30; G16H 50/20; G16H 50/70

(57) ABSTRACT

Developmental outcomes for preterm infants can be forecast using an automated classifier. The automated classifier can be trained to predict a result of a particular developmental assessment using neuroimaging data collected from preterm infants that measures anatomical and/or functional characteristics of the nervous system, including cortical and/or subcortical brain structures, and developmental The classifier can enable forecasting of developmental problems that have not yet been observed, based on structure and/or function of the preterm infant's brain, and the forecast can be used to inform decisions regarding developmental interventions.

### CROSS-REFERENCE TO RELATED APPLICATION

This application claims the benefit of U.S. Provisional Application No. 63/461,204, filed Apr. 21, 2023, which is incorporated herein by reference in its entirety.

### BACKGROUND

The present disclosure relates generally to predicting developmental outcomes in preterm children and in particular to using neural data collected in infancy to forecast future developmental outcomes in preterm children.

Preterm births, i.e., births that occur before 37 weeks of gestation, are estimated to constitute more than 10% of all live births worldwide. Although improvements in perinatal care have contributed substantially to an increase in the survival rate, studies have shown that preterm infants continue to lag behind their peers who are born at full term across multiple developmental domains, including socioemotional, cognitive, language, and motor development. Although some preterm infants are born with life-threatening complications such as brain hemorrhage, the majority are born without any such problems. Without any observable medical complications or other traits, it is difficult to predict which preterm infants will experience developmental disorders as they progress through childhood. Therefore, unless the child is born very preterm (e.g., before 32 weeks of gestation) or has significant medical complications, the standard of care is to wait until developmental problems become observable before intervention. This could mean waiting until preschool years or school age, missing the earliest window of opportunity when early intervention is most likely to be effective.

Gross pathologies, such as periventricular leukomalacia and intraventricular hemorrhage, have been used to identify neurological deficits in preterm infants. Such pathologies can indicate likely developmental problems in socioemotional, cognitive, language and motor domains; however, only a minority of preterm infants who experience developmental problems exhibit gross pathologies of this kind. For preterm infants born before 32 weeks of gestation, more advanced neurological techniques have been used to identify prognostic indicators of development; however, this population is more likely to show signs of developmental deficits.

### SUMMARY

Certain embodiments of the invention relate to systems and methods for predicting, or forecasting, developmental outcomes for preterm infants, including preterm infants born at a gestational age between 32 and 37 weeks who are otherwise healthy. This group constitutes the majority of the preterm population and is also the population whose developmental outcomes are the most difficult to predict using existing methodologies. Various embodiments incorporate neuroimaging to obtain anatomical and/or functional measurements of the nervous system, including not only structures of the telencephalon (much of the “cortical” brain), but also the diencephalon, medulla, pons, midbrain and other structures of the nervous system.

Neuroimaging can incorporate a number of different methodologies, and techniques, including magnetic resonance imaging (MRI), which can be structural or functional; electroencephalography (EEG); magnetoencephalography (MEG); functional near infrared stimulation (fNIRS); and so on. Neuroimaging data for a child can be collected in infancy (e.g., at age up to 3 months) and used to extract quantitative data characterizing structures and/or functions of the nervous system, including cortical and/or subcortical regions. The quantitative data can be provided as input to an automated classifier that has been trained to predict a result of a developmental assessment performed at a later age (e.g., an age of 1 year, 18 months, 2 years, 6 years, etc.). Based at least in part on the predicted result, a developmental outcome can be forecast for the child. The forecast outcome can in turn be used to inform decisions regarding interventions aimed at fostering development. A number of developmental domains can be considered, including emotional and self-regulation, social interaction, language and communication, motor, and/or cognition, and different classifier models can be trained to make predictions in different domains.

The following detailed description, together with the accompanying drawings, will provide a better understanding of the nature and advantages of the claimed invention.

### BRIEF DESCRIPTION OF THE DRAWINGS

FIG. 1 shows a flow diagram of a process for training and using an automated classifier to predict developmental outcomes for individual children according to an embodiment of the present invention.

FIGS. 2 and 3 show example images of brain areas with highlighting of regions predictive of ASQ-3 fine motor scores for preterm children according to some embodiments.

FIG. 4 shows example images of brain areas with highlighting of regions predictive of ASQ-3 personal-social scores for preterm children according to some embodiments.

FIG. 5 is a graph showing performance of a classifier predicting language development in preterm children according to some embodiments.

### DETAILED DESCRIPTION

The following description of exemplary embodiments of the invention is presented for the purpose of illustration and description. It is not intended to be exhaustive or to limit the claimed invention to the precise form described, and persons skilled in the art will appreciate that many modifications and variations are possible. The embodiments have been chosen and described in order to best explain the principles of the invention and its practical applications to thereby enable others skilled in the art to best make and use the invention in various embodiments and with various modifications as are suited to the particular use contemplated.

According to various embodiments, developmental outcomes for preterm infants can be forecast using an automated classifier. The automated classifier can be trained to predict a result of a particular developmental assessment using neuroimaging data collected from preterm infants that measures anatomical and/or functional characteristics of the central nervous system, including cortical and/or subcortical brain structures, and developmental assessment results obtained at a later stage of the child's development. The classifier can enable forecasting of developmental problems that have not yet been observed, based on structure and/or function of the preterm infant's central nervous system, and the forecast can be used to inform decisions regarding developmental interventions.

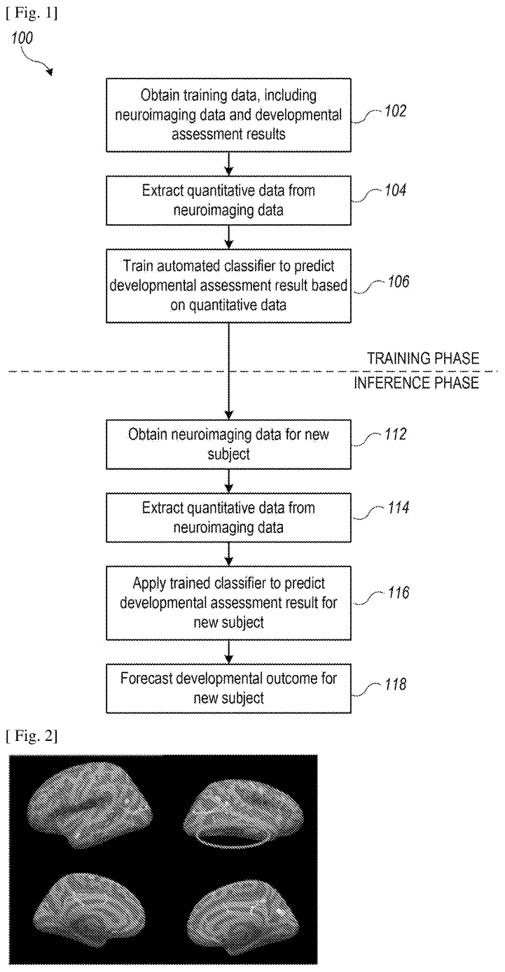

FIG. 1 shows a flow diagram of a process 100 for training and using an automated classifier to predict developmental outcomes for individual children according to an embodiment of the present invention. Process 100 can be implemented using a suitably programmed computer system. Blocks 102-106 correspond to a training phase; blocks 112-118 correspond to a testing (or inference) phase.

At block 102, a training data set is obtained. The training data set can include neuroimaging data and developmental assessment results. The neuroimaging data can be obtained from children near the time of birth (e.g., in the first three months after birth or the first six months after birth; in some embodiments, the time frame can be adjusted to account for gestational age at birth). Developmental assessment results can be obtained at a later time (e.g., at a later age such as one year, 18 months, 2 years, 6 years, or 12 years). In some embodiments, the training data can include data from preterm infants as well as full-term infants (gestation period of at least 38 weeks); alternatively, the training data can include data only from preterm infants, in particular preterm infants born at gestational ages between 32 and 37 weeks or between 32 and 39 weeks.

Neuroimaging data can include any combination of medical images and/or other data characterizing structure and/or function of the child's neural centers, including cortical and/or subcortical neural centers, and/or neural pathways. Examples of neuroimaging data include magnetic resonance (MR) images, functional MR images, electroencephalogram (EEG), magnetoencephalogram (MEG), functional near-infrared spectroscopy (fNIRS), and so on.

Assessment of developmental levels can be performed at various ages and can use a variety of assessment instruments. Developmental assessment can focus on one or more domains, including emotion and self-regulation, social interaction, language and communication, motor skills, and/or cognition. For example, the Ages & Stages Questionnaires Third Edition (ASQ-3) is a well-known tool designed to identify delays in communication skills, gross motor skills, fine motor skills, problem solving skills, and personal social skills in children from 2 to 66 months. As another example, language development for Chinese-learning infants can be assessed using tools provided by the Chines Communicative Development Initiative. A variety of tests or suites of assessments may be used, depending on the particular developmental domain (s) of interest, the child's first language, the child's age and other considerations.

At block 104, quantitative data can be extracted from the neuroimaging data. The quantitative data can include data characterizing various structures and/or functions of the central nervous system. For example, quantitative data can characterize one or more structural features such as morphology, volume, and/or thickness of a brain structure (including cortical and/or subcortical structures) or other structure of the central nervous system; integrity of white matter tracks in a brain structure or other structure of the central nervous system; and/or characteristics of localized and/or distributed nerve centers across neural pathways. In addition or instead, quantitative data can characterize one or more functional features such as functionality along primary and/or associational neural pathways; functionality of the brainstem nucleus and connected neural pathways; and/or functionality of the thalamic nucleus and connected neural pathways.

In some embodiments, additional quantitative data can be obtained by comparing two sets of neuroimaging data to quantify differences. For example, structural data of the subject child can be compared to structural data for healthy full-term infants. As another example, functional data for the subject child at rest can be compared to functional data for the same child responsive to a stimulus. Functional data for the subject child responsive to different stimuli can be compared. Functional data for the subject child can also be compared to functional data for healthy full-term infants. Inputs to the automated classifier can include any or all of the above or other quantitative data obtained from neuroimaging data.

At block 106, an automated classification algorithm (also referred to as a “classifier”) can be trained using the training data set. Suitable algorithms include machine-learned classification algorithms such as a Support Vector Machine (SVM) or ranking SVM (RankSVM), a Support Vector Regression classifier (SVR), a generalized linear model, or other classification algorithms.

SVM is a machine-learned classification technique that takes as input a feature vector in a space of arbitrary dimension and a binary classification and maps the feature vector to a point in a classification space such that a hyperplane in the classification space (referred to as a “margin”) separates the points corresponding to the (binary) classification of the corresponding feature vector. SVR is a type of SVM that can produce a continuously-valued output. In most SVM implementations, the margin may be a “soft margin,” allowing the classification to be less than 100% accurate. In the present context, the feature vector can include features extracted from the quantitative data for a given child, and the binary classification (for SVM) can be “low probability” or “high probability” of a developmental problem based on comparing the child's score on a developmental assessment to a threshold.

RankSVM is a machine-learned classification technique whose goal is to construct ordered models that can be used to sort unseen data according to their degree of relevance or importance. RankSVM can be used to form ranking models by minimizing a regularized margin-based pairwise loss. RankSVM uses SVM to compute a weight vector that maximizes the difference of data pairs in ranking. In principle, RankSVM requires investigating every data pair as potential candidates for support vectors, and the number of data pairs is quadratic to the size of the training set. In practice, this can result in low computational efficiency for large training sets and/or large feature vectors. Accordingly, optimizations may be employed to increase computational efficiency; specific examples are described below.

A Generalized Linear Model is a regression-based classification technique that allows for additional flexibility compared to linear regression. In particular, the input variables can have non-normal distributions such that the variance of each measurement can be a function of the predicted value, and the linear model can be related to the response variable by a link function corresponding to the non-normal distribution. Parameters of the model can be trained using machine-learning techniques.

Regardless of the particular algorithm, the classifier can be trained to predict results of one or more particular developmental assessments based on neuroimaging data. The prediction can be qualitative (e.g., low or high likelihood of an assessment score being in a range that indicates a developmental problem) or quantitative (e.g., a predicted assessment score or a quantitative probability that the assessment score will indicate a developmental problem). In some embodiments, a threshold on the assessment score can be used to define whether a developmental problem is considered present. For instance, assessment scores in the lowest quartile may be considered to indicate a problem. Other thresholds can be used. In embodiments where an aim is to guide decisions about preemptive intervention, thresholds can be defined based on whether the severity of the problem would warrant intervention.

In some instances, the classifier can be trained using a developmental assessment performed at a particular age (e.g., 18 months, 2 years, 6 years) or within a particular age range (e.g., 18 months to 2 years or 5 to 6 years or the like). Where this is the case, a predicted result can be considered as age-specific (e.g., probability of motor-skill problems being observed at age 2 years). As noted above, training can be based on data obtained from children whose developmental status has been assessed in the age range of interest and for whom relevant neuroimaging data was obtained in early infancy (e.g., in the first three months or first six months).

It should be understood that many combinations of classifier algorithm, neuroimaging data, quantitative data extracted from the neuroimaging data, and developmental assessment instruments can be used. The neuroimaging data can include any data indicative of structural and/or functional integrity of the child's nervous system along cortical and subcortical pathways. For example, in various embodiments, neuroimaging data can include any combination of: (1) structural data characterizing anatomical details of the child's subcortical and/or cortical neural centers, which can be obtained using MRI or other imaging techniques; (2) functional data characterizing subcortical and/or cortical responses when the child is at rest (e.g., during sleep); and/or (3) functional data characterizing subcortical and/or cortical responses to external sensory stimulation (e.g., sounds or sights). Functional data can be obtained using various techniques such as fMRI, EEG, MEG, or fNIRS.

The trained classifier can thereafter be used to predict developmental assessment results for new (previously unseen) subject. At block 112, neuroimaging data can be obtained for the new subject. The neuroimaging data can be of the same kind that was obtained for training subjects at block 102 and can be obtained at the same (approximate) age. At block 114, quantitative data can be extracted from the neuroimaging data; the same data types and extraction techniques used at block 104 can be applied. At block 116, the trained classifier can be used to predict a developmental assessment result for the new subject based on the quantitative data extracted at block 114. As noted above, the predicted result can be qualitative or quantitative, depending on the particular classifier model and how the classifier was trained. At block 118, a developmental outcome for the new subject can be forecast, based at least in part on the predicted developmental assessment result obtained at block 114.

In some embodiments, the forecast outcome can be the predicted developmental assessment result. If desired, additional information can be incorporated to refine or modify the result. For instance, multiple models may be trained to predict developmental assessment results (e.g., using different assessment instruments, different developmental domains, and/or different input data), and the forecast can incorporate predictions from multiple models. The forecast outcome can be used to inform decisions regarding whether to engage in preemptive intervention (and what form of intervention) to facilitate the child's development and/or mitigate future developmental problems.

In some embodiments, the classifier assumes a baseline general health status for the child (e.g., no evidence of gross pathologies). If desired, classifiers can be trained for separate categories of general health, and a predicted result for a particular child can be generated by selecting the appropriate trained classifier according to the child's general health status. Baseline health status can be established by direct examination of the child, or by report (e.g., from a caregiver).

To illustrate the effectiveness of this approach, specific example implementations and results will now be described. It should be understood that embodiments of the invention are not limited to these examples.

### Example 1

In one implementation, T1-weighted MRI neuroanatomical data was used to forecast developmental outcomes as measured by ASQ-3.

Sixty-one infants born in the moderate preterm to early term range underwent structural MRI while naturally sleeping within the first three months of age (corrected for prematurity), to produce T1-weighted MRI neuroanatomical data. Developmental outcomes were measured by ASQ-3.

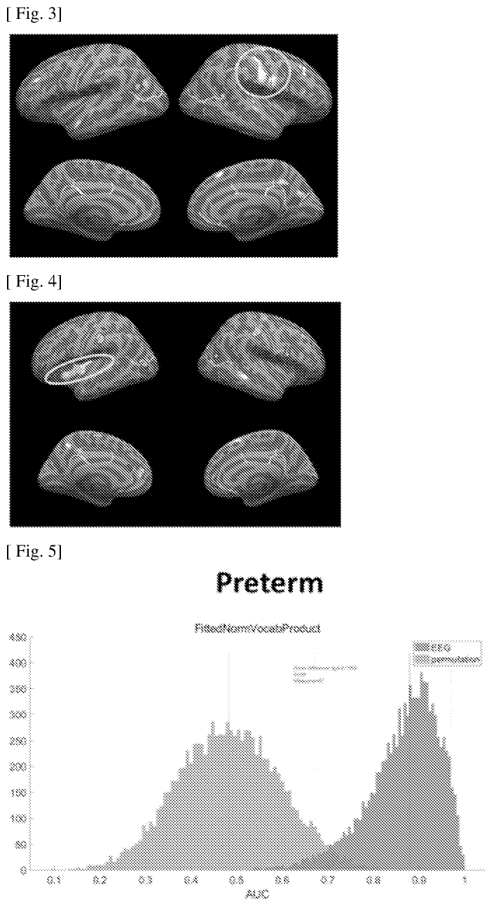

Generalized Linear Models with sex, postnatal age or group (preterm vs term) controlled were built to assess the whether cortical thickness data can predict developmental outcomes across different domains. It was found that cortical thickness in the right inferior temporal cortex and right postcentral cortex significantly predicted ASQ-3 fine motor scores. FIGS. 2 and 3 show example images of brain areas with highlighting of predictive regions. In FIG. 2, cortical thickness of the inferior temporal region was negatively correlated with the ASQ-3 fine motor score, with corrected p=0.0378 and cluster size of 1304.37 mm². Peak p (−logP)=4.0231 is located at coordinate (54.5, −31.3, −26.9). In FIG. 3, cortical thickness of the post central region was positively correlated with the ASQ-3 fine motor score, with corrected p=0.0032 and cluster size of 1891.34 mm². Peak p (−logP)=2.9872 is located at coordinate (56.3, −11.7, 36.9).

It was also found that cortical thickness in the left insular and opercular part of the inferior frontal cortex predicted personal-social scores. FIG. 4 shows example images of brain areas with highlighting of predictive regions. Cortical thickness of the insular and pars opercularis regions was positively correlated with the ASQ-3 personal-social score, with corrected p=0.0026 and cluster size of 1877.82 mm². Peak p (−logP)=3.3019 is located at coordinate (−39.5, 6.5, 11.7).

### Example 2

In another implementation, short-and long-latency neural responses to auditory speech stimulation collected via EEG were used to forecast language developmental outcomes as measured by the Chinese Communicative Development Inventories (CCDI) for Chinese-learning preterm infants.

EEG data were collected from preterm infants as young as 0.1 month of age and language developmental data were collected up to 16 months of age. SVR classification with cross-validation was conducted to classify 58 preterm infants based on their CCDI levels, at or below 25th percentile or above 25th percentile, using the EEG and baseline health status data as input. FIG. 5 is a graph showing performance of SVR measured as AUC, or area under the receiver operating characteristic (ROC) curve. In this implementation, the SVR-ROC AUC was 0.865 (p<0.001) and sensitivity was 92% (p<0.05) with bootstrap-permutation.

### Computer Implementations

Data analysis and computational operations of the kind described herein can be implemented in computer systems that may be of generally conventional design. Such systems may include one or more processors to execute program code (e.g., general-purpose microprocessors usable as a central processing unit (CPU) and/or special-purpose processors such as graphics processors (GPUS) that may provide enhanced parallel-processing capability); memory and other storage devices to store program code and data; user input devices (e.g., keyboards, pointing devices such as a mouse or touchpad, microphones); user output devices (e.g., display devices, speakers, printers); combined input/output devices (e.g., touchscreen displays); signal input/ output ports; network communication interfaces (e.g., wired network interfaces such as Ethernet interfaces and/or wireless network communication interfaces such as Wi-Fi); and so on. Building and testing of classifiers as described herein (including linear SVM, nonlinear SVM, and RankSVM classifiers) can be supported using existing application software such as MATLAB or custom-built application software. Such software may be said to configure the processor to perform various operations, including operations described herein.

Computer programs incorporating various features of the present invention may be encoded and stored on various computer readable storage media; suitable media include magnetic disk or tape, optical storage media such as compact disk (CD) or DVD (digital versatile disk), flash memory, and other non-transitory media. (It is understood that “storage”of data is distinct from propagation of data using transitory media such as carrier waves.) Computer readable media encoded with the program code may be packaged with a compatible computer system or other electronic device, or the program code may be provided separately from electronic devices (e.g., via Internet download or as a separately packaged computer-readable storage medium).

In alternative embodiments, a purpose-built processor may be used to perform some or all of the operations described herein. Such processors may be optimized, e.g., for performing computations to train an SVM classifier, and may be incorporated into computer systems of otherwise conventional design or other computer systems.

### Additional Embodiments

As described above, embodiments of the invention provide machine-learning techniques that can be used to train an automated classifier to predict a developmental outcome for a preterm infant based on neuroimaging data representing structure and/or function of the infant's central nervous system or portions thereof (e.g., the brain, brainstem, or specific brain area). Depending on the particular classifier, the prediction can be binary (e.g., developmental assessment score above or below some threshold) or quantitative (e.g., developmental assessment score within one of three or more ranges). The prediction can be used to inform decisions regarding whether and when to begin specific interventions to address developmental challenges, although it is expected that such decisions may also consider other factors as well.

While the invention has been described with reference to specific embodiments and examples, those skilled in the art with access to the present disclosure will recognize that variations and modifications are possible. For instance, examples described above were based on data from preterm infants with a gestation period of 32 to 37 weeks. However, it is expected that similar processes could also be applied to other groups of children, e.g., such as preterm infants with shorter gestation periods. (“Gestation period” as used herein can be measured in the standard manner, relative to the first date of the mother's most recent menstrual cycle, or using more accurate techniques if desired.) Age of the child can be defined based on birth date; in some embodiments, the age can be adjusted to account for prematurity.

Neuroimaging data characterizing neurological structure and/or function may be collected using a number of techniques. For structural data, techniques include but are not limited to MRI. As noted above, MRI images can include anatomical and/ or functional images, and some or all scans may be performed with the child under sedation or general anesthesia, awake, or in natural sleep. Other imaging techniques may be used to produce neurological data characterizing brain composition (or composition of other structures of the central nervous system) in specific locations, including characterizations based on local tissue density (e.g., VBM), similarity in local spatial morphological patterns (e.g., MVPS) for gray matter and/or white matter. Functional and/or neurophysiological profiles can be obtained using functional MRI (e.g., to measure neural hemodynamic responses), electrophysiological responses measured by EEG or MEG, and so on. In some instances, including examples described above, neuroimaging data may be acquired while the child is not receiving any external stimulation, which can be with the child awake but at rest, in natural sleep, or under sedation (including general anesthesia). In some embodiments, neuroimaging data may be acquired while the child is receiving external stimulation. As noted, neuroimaging data acquired with and without stimulation and/ or neuroimaging data acquired with different stimuli can be compared to provide additional quantitative data as input to the classifier.

Various structures and/or functions within a child's central nervous system can be quantified using the neuroimaging data, including cortical and/or subcortical structures. Examples of structural features of interest include: morphology, volume, and/or thickness of a brain structure or other structure in the central nervous system; integrity of white matter tracks in a brain structure or other structure in the central nervous system; characteristics of localized and/or distributed nerve centers across neural pathways. Examples of functional features of interest include: functionality along primary and/or associational neural pathways; functionality of the brainstem nucleus and connected neural pathways; and functionality of the thalamic nucleus and connected neural pathways.

Developmental assessment can be based on any age-appropriate instrument for measuring development quantitatively or qualitatively, not limited to specific examples described above. Development can be assessed in a number of different domains, including emotion and self-regulation, social interaction, language and communication, motor skills, and/or cognition. Developmental assessment can be performed at any age. For example, the model can be trained based on developmental assessments of children up to 18 months, or up to 12 years, or some other specified age range. Once trained, the model can be applied to make predictions for children in the same age range that was used during training. It should be understood that the neuroimaging data can be obtained at one age (e.g., up to 3 months after birth) while developmental assessments are performed at a later age (e.g., 18 months, 2 years, 6 years, 12 years). In some embodiments, multiple classifier models can be trained using developmental assessments at different ages to provide longitudinal predictions of when developmental problems are most likely to emerge or whether a child whose development is initially delayed is likely to “catch up” to peers.

A variety of classifiers (machine-learned algorithms that can be trained to predict an outcome for an unseen data sample based on a set of data samples with known outcomes) can be used. In some instances, a linear or non-linear SVM provides an effective binary classifier that can be used to indicate whether the child is likely to experience significant developmental problems. A RankSVM may allow a more quantitative prediction of the severity of any problems, e.g., where development is measured on an ordinal (non-continuous) scale. In embodiments where outcomes are measured on a continuous scale, other classifiers such as Support Vector Regression (SVR) may be used to provide quantitative predictions of the severity of developmental problems. Other classifiers can be based on other statistical techniques, such as random forest methods, Bayesian inferencing, or univariate or multivariate analytics. Still other algorithms, such as Hidden Markov Model, and deep learning algorithms (e.g., artificial neural networks) may be substituted. Those skilled in the art with the benefit of this disclosure will be able to implement additional embodiments using these and other classifiers. The parameters used for training and testing the classifier may be varied, including the size of training data sets and the particular combination of inputs. A particular algorithmic implementation of training is not required.

Predicted outcomes generated in the manner described herein may be used in treatment planning. For example, the predicted outcome may inform the decision whether to proceed with particular interventions for a given child aimed at preventing or mitigating developmental problems.

Thus, although the invention has been described with respect to specific embodiments, it will be appreciated that the invention is intended to cover all modifications and equivalents within the scope of the following claims.

1. A method of predicting a developmental outcome for a child born prematurely, the method comprising:
determining a baseline health status of the child;
obtaining neuroimaging data characterizing one or both of anatomical or functional properties of the child's central nervous system;
extracting quantitative data from the neuroimaging data;
determining a predicted result of a developmental assessment for the child by analyzing the quantitative data using a statistically-validated classifier that has been trained to predict a result of the developmental assessment for an individual, wherein the training of the classifier is based on corresponding quantitative data from a training data set obtained from a plurality of previous preterm children having known developmental assessment results; and
generating a predicted developmental outcome for the child based on the predicted result of the developmental assessment and the baseline health status of the child.

2. The method of claim 1 wherein the developmental assessment pertains to one or more of emotion and self-regulation, social interaction, language and communication, motor skills, or cognition.

3. The method of claim 1 wherein the child and the previous preterm children are born at a gestational age between 32 and 39 weeks.

4. The method of claim 1 wherein the training data set includes neuroimaging data obtained for the previous preterm children at an age of up to six months and developmental assessment results obtained for the previous preterm children at an age of at least one year.

5. The method of claim 1 wherein the child is under 18 months of age and the corresponding quantitative data was obtained from previous preterm children who were under 18 months of age.

6. The method of claim 1 wherein the child is under 12 years of age and the corresponding quantitative data was obtained from previous preterm children who were under 12 years of age.

7. The method of claim 1 wherein the neuroimaging data includes magnetic resonance (MR) images characterizing anatomical properties of the child's brain.

8. The method of claim 1 wherein the neuroimaging data includes data characterizing functional properties of the child's brain and is obtained using one or more of electroencephalography (EEG), magnetoencephalography (MEG), functional near-infrared spectroscopy (fNIRS), or functional magnetic resonance imaging (fMRI).

9. The method of claim 1 wherein the neuroimaging data includes data acquired while the child is sedated.

10. The method of claim 1 wherein the neuroimaging data includes data acquired while the child is in natural sleep.

11. The method of claim 1 wherein the quantitative data includes data characterizing one or more of morphology, volume, or thickness of one or more regions of the central nervous system.

12. The method of claim 1 wherein the quantitative data includes data characterizing integrity of white matter tracks in one or more regions of the central nervous system.

13. The method of claim 1 wherein the quantitative data includes data characterizing structures along one or more cortical or subcortical pathways.

14. The method of claim 1 wherein the quantitative data includes data characterizing a function along a neural pathway.

15. The method of claim 14 wherein the quantitative data includes data characterizing a function of a processing center of a sensory neural pathway.

16. The method of claim 1 wherein the quantitative data includes data characterizing a function of a brainstem nucleus and a connected pathway.

17. The method of claim 1 wherein the quantitative data includes data characterizing a function of a thalamic nucleus and a connected pathway.

18. The method of claim 1 wherein the quantitative data includes functional data characterizing a cortical or subcortical response to a stimulus.

19. The method of claim 1 wherein the quantitative data includes functional data characterizing a cortical or subcortical response when at rest.

20. The method of claim 1 wherein obtaining the neuroimaging data includes:
obtaining first functional data characterizing a cortical or subcortical response to a stimulus; and
obtaining second functional data characterizing the cortical or subcortical response when at rest,
wherein the quantitative data includes data indicating a difference between the first functional data and the second functional data.

21. The method of claim 1 wherein obtaining the neuroimaging data includes:
obtaining first functional data characterizing a cortical or subcortical response to a first stimulus; and
obtaining second functional data characterizing the cortical or subcortical response to a second stimulus different from the first stimulus,
wherein the quantitative data includes data indicating a difference between the first functional data and the second functional data.

22. The method of claim 1 wherein extracting the quantitative data includes comparing the neuroimaging data to baseline data associated with children born at term.

23. The method of claim 1 wherein the statistically-validated classifier is a support vector machine (SVM).

24. The method of claim 23 wherein the predicted likelihood from the SVM is either a high degree or a low degree.

25. The method of claim 23 wherein the predicted likelihood from the SVM is a continuously-valued output.

26. The method of claim 1 wherein the statistically-validated classifier is based on random forest methods.

27. The method of claim 1 wherein the statistically-validated classifier is based on Bayesian inferencing.

28. The method of claim 1 wherein the statistically-validated classifier is based on univariate or multivariate analytics.

29. A system comprising:
a memory; and
a processor coupled to the memory and configured to perform a method according to any one of claims 1 to 28.

30. A computer-readable storage medium having stored therein program code instructions that, when executed by a processor in a computer system, cause the processor to perform a method according to any one of claims 1 to 28.
